(19) United States
(12) Patent Application Publication
(10) Pub. No.: US 2013/0289637 A1
(43) Pub. Date: Oct. 31, 2013

(54) ELECTROMAGNETIC INTERFERENCE SHIELDING FOR USE WITH AN IMPLANTABLE MEDICAL DEVICE INCORPORATING A RADIO TRANSCEIVER

(71) Applicants: Jorge Amely-Velez, Simi Valley, CA (US); Kavous Sahabi, Winnetka, CA (US); Anthony Li, Valencia, CA (US); Katerina Serafimova, Granada Hills, CA (US); Ana Maria Gonzalez Nieto, Stockholm (SE); Ting Jun Lo, Kista (SE); Daniel Thomas, Jarfalla (SE); Jeffrey Alves, Belmont, CA (US)

(72) Inventors: Jorge Amely-Velez, Simi Valley, CA (US); Kavous Sahabi, Winnetka, CA (US); Anthony Li, Valencia, CA (US); Katerina Serafimova, Granada Hills, CA (US); Ana Maria Gonzalez Nieto, Stockholm (SE); Ting Jun Lo, Kista (SE); Daniel Thomas, Jarfalla (SE); Jeffrey Alves, Belmont, CA (US)

(73) Assignee: PACESETTER, INC., Sylmar, CA (US)

(21) Appl. No.: 13/458,934

(22) Filed: Apr. 27, 2012

(51) Int. Cl. A61N 1/375; A61N 1/39

(52) U.S. Cl. 607/5; 607/72; 607/60

(57) ABSTRACT

The implantable medical device includes high-voltage components (such as defibrillation shock generation components) operative to generate high-voltage pulses for delivery to tissues of the patient while using the case or housing of the device as a stimulation electrode. The device also includes low-voltage Medical Implant Communication Service (MICS) or Medical Device Radiocommunications Service (MedRadio) components operative to generate low-power signals for communicating with an external device via radio frequencies while using the case as part of an antenna. A conductive noise shield is mounted within the case of the device and interposed between the high-voltage components and the case, with the shield configured to attenuate electrical interference between the high-voltage components and the case to facilitate radio-frequency communication between the low-voltage MICS/MedRadio components and the external device, which use the case as part of the antenna.

### FIELD OF THE INVENTION

The invention generally relates to implantable medical devices, such as implantable cardioverter/defibrillators (ICDs), and in particular to electromagnetic interference (EMI) shielding techniques for improving Medical Implant Communication Service (MICS) radio transmissions or Medical Device Radiocommunications Service (MedRadio) transmissions.

### BACKGROUND OF THE INVENTION

ICD is an implantable medical device equipped to detect and treat atrial fibrillation (AF) and/or ventricular fibrillation (VF) and deliver electrical shocks to terminate VF. Typically, the metal case or housing (or “can”) of the ICD is used as a shocking electrode along with a coil electrode implanted on or in the heart of the patient. Various high-voltage components within the ICD (such as switched mode power supplies and high-voltage charging circuits) operate to charge a shocking capacitor so that it can deliver a powerful defibrillation shock to the heart of the patient in response to VF. At least some state-of-the-art ICDs are also being equipped with MICS-band or MedRadio-band communication devices for communicating with external programming units or systems such as bedside monitors to relay patient diagnostic data. (Note that in 2009 the United States Federal Communications Commission (FCC) adopted rules for the creation of MedRadio to replace MICS. MedRadio maintains the spectrum previously allocated for MICS (402-405 MHz) while adding additional adjacent spectrum (401-402 MHz and 405-406 MHz). Herein, the term “MICS/MedRadio” will be used for the sake of completeness and generality to refer to MICS, MedRadio or both.) The MICS/MedRadio-band devices use the case of the ICD as part of an antenna for transmitting radio signals. However, EMI from the high-voltage components of the ICD can interfere with the relatively low power MICS/MedRadio transmissions, thereby hindering or preventing communication.

In general, it is not recommend using antenna elements in association with circuitry not related to radio-frequency (RF) transmission and reception. However, due to size restrictions and miniaturization concerns within ICDs, medical device designers may need to combine certain high-voltage circuit components with elements of low-voltage circuits in order to achieve a viable “loosely coupled” system. These compromises can result in less than ideal performance, especially with ICD-based RF systems where signal levels are only on the order of microvolts. Accordingly, it would be desirable to address noise interference problems arising between the high-voltage charging and shocking components of an ICD and the relatively low-voltage MICS/MedRadio radio components, which use the device case as a ground plane for its antenna.

### SUMMARY OF THE INVENTION

In accordance with an exemplary embodiment of the invention, an implantable medical device is provided for implant within a patient wherein the device includes high-voltage defibrillation components operative to generate high-voltage pulses for delivery to tissues of the patient while using the case of the device as a stimulation electrode. The device also includes low-voltage components (such as a MICS/MedRadio transceiver) operative to generate low-power signals for communicating with an external system while using the case as part of an antenna. A conductive EMI noise shield is mounted within the case and conformably interposed between the high-voltage components and the inner surface of the case, with the shield configured to attenuate electrical interference from the high-voltage components to facilitate communication between the low-power components and the external system, which use the case as part of the antenna.

In an illustrative embodiment, the implantable medical device is an ICD having a charge-storage capacitor operative to store a high-voltage charge for use in generating a defibrillation shock. A high-voltage circuit having one or more diodes, transformers or switched-mode power supplies is provided to charge the capacitor using batteries or other power sources. The high-voltage components (particularly the switched mode power supply) are configured to operate in the low kilohertz (KHz) region to charge the capacitor. Rectified diodes on the secondary side of the transformer produce first order harmonics at twice the low operating frequency of the charger. The low-voltage components include MICS/MedRadio radio components that operate using intermediate frequency (IF) signals that correspond to twice the fundamental charger frequency (and hence might detect unwanted harmonics produced by the high voltage components of the charger if no shield were provided.) The MICS/MedRadio radio components are coupled to the device case to use the case as a ground plane for the antenna for communicating with the external systems using RF signals. That is, the ground plane of the antenna (the device case) is shared by the MICS/MedRadio components and the high voltage shocking components, with high voltage capacitors and diodes only a few semiconductor components away from the case. To attenuate interference between the high-voltage components and the ground plane of the antenna that might hinder or interfere with MICS/MedRadio communications, the device includes a thin conformal copper shield configured to have high impedance relative to electrical signals generated by the high-voltage components so as to significantly attenuate electrical interference between the high-voltage components and the case. However, the noise shield has relatively low impedance relative to magnetic signals generated by the high-voltage components and does not significantly attenuate the magnetic signals.

In this regard, if EMI noise is produced by a mechanism that generates a large current under low potential conditions, it is known as a magnetic or low impedance source due to the small ratio of the electric (E) to magnetic (H) components. If the noise source operates at high voltage with small amounts of currents flowing, the E to H ratio is high and the source is known as an electric field source. The aforementioned conformal shield is configured to attenuate noise from the high-voltage components by exploiting impedance discontinuities between the noisy high-voltage components and the shield. Within an ICD, the source of noise is mostly electrical in nature. Due to the thinness and permeability of the copper metal employed by the conformal shield (e.g. about 1-2 mils thick), the shield provides very little attenuation to magnetic noise sources because the low impedance of the magnetic waveform closely matches the low impedance of the copper material. In other words, closely matched impedances allow the magnetic noise source waves to pass through the copper metal with very little attenuation. However, the electric field wave has very high impedance compared to the shield metal and so much of the incident waveform is reflected and prevented from reaching the antenna ground plane.

In one particular example, the conductive shield has a first shielding portion near an interior surface of a first portion of the case via a non-conducting adhesive. The conductive shield also has a second shielding portion near an interior surface of a second portion of the case and connected to a lowest potential ground plane of the high-voltage components of the device via a conductive adhesive or other suitable electrical contact. The conductive adhesive is a pressure sensitive adhesive that includes spherically-shaped beads configured to enhance contact between the shield and the ground plane of the high-voltage components. Both portions of the shield have an insulating layer interposed between an inner surface of the shield and various internal electrical components of the device such as one or more batteries, diodes and high voltage capacitors to help insulate those components and prevent electrical shorts.

With this exemplary configuration, to deliver a high voltage shock, one terminal of the shocking capacitor (such as the anode) is connected via internal switches to an electrical feed-through terminal via a right ventricular (RV) lead to a coil electrode implanted in the RV. The other terminal of the shocking capacitor (such as the cathode) is connected to the interior surface of the device case. In this manner, the RV coil and the device case are used as opposing anodes/cathodes for delivery of the high voltage shock. To transmit data via MICS/MedRadio bands, the high voltage components can remain connected to the device case (i.e., the high voltage components need not be disconnected from the case; rather the radio and high voltage components can operate simultaneously.) The MICS/MedRadio components are always connected to the case (via one or more capacitors) to use the case as part of its antenna (along with a radiating wire mounted inside a header of the device.) With this configuration, the noise shield does not form a part of the MICS/MedRadio transmission circuit. Instead, the noise shield is positioned to attenuate radiated EMI noise (particularly the aforementioned electrical components thereof) between the high-voltage components of the ICD and the device case so the noise does not significantly interfere with MICS/MedRadio communications, which use the case as part of the antenna. Note that with this configuration, the case is always part of the antenna. The case thereby supports a dual function of being a stimulation electrode and an antenna component because there are coupling capacitors between the MICS/MedRadio transceiver and the antenna (wherein the antenna comprises the case plus the radiating element in the header.) Meanwhile, the shield does not carry any current.

System and method examples of various embodiments of the invention are described herein.

### BRIEF DESCRIPTION OF THE DRAWINGS

The above and further features, advantages and benefits of the invention will be apparent upon consideration of the present description taken in conjunction with the accompanying drawings, in which:

FIG. 1 illustrates pertinent components of an implantable medical system having an ICD equipped for MICS/MedRadio communication with an external system and incorporating an internal EMI noise shield;

FIG. 2 is a perspective view of the ICD of FIG. 1 shown without its case and particularly illustrating the EMI noise shield;

FIG. 3 is another perspective view of the ICD of FIG. 1, also illustrating the EMI noise shield;

FIG. 4 is a planar view of an alternative configuration of the EMI noise shield of FIGS. 2 and 3, shown flat before assembly;

FIG. 5 is a planar view of another configuration of the EMI noise shield of FIGS. 2 and 3;

FIG. 6 is a schematic illustration of pertinent components of the ICD of FIG. 1, particularly identifying certain conduction pathways;

FIG. 7 illustrates exemplary techniques pertaining to assembly and usage of the ICD of FIG. 1;

FIG. 8 illustrates exemplary techniques of FIG. 7 in greater detail;

FIG. 9 is a simplified, partly cutaway view, illustrating the ICD of FIG. 1 along with a set of leads implanted on or in the heart of the patient; and

FIG. 10 is a functional block diagram of the ICD of FIG. 9, illustrating basic circuit elements that provide cardioversion, defibrillation and/or pacing stimulation in the heart, as well as components for MICS/MedRadio communication.

### DETAILED DESCRIPTION OF THE PREFERRED EMBODIMENTS

The following description includes the best mode presently contemplated for practicing the invention. This description is not to be taken in a limiting sense but is made merely to describe general principles of the invention. The scope of the invention should be ascertained with reference to the issued claims. In the description of the invention that follows, like numerals or reference designators will be used to refer to like parts or elements throughout.

### Overview of Implantable System

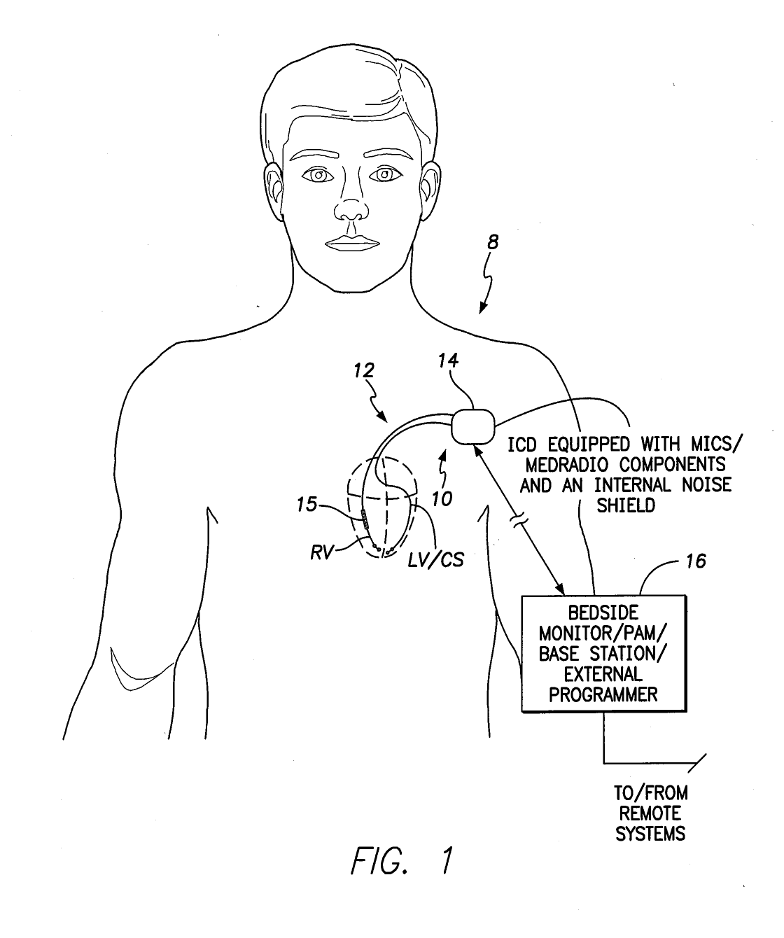

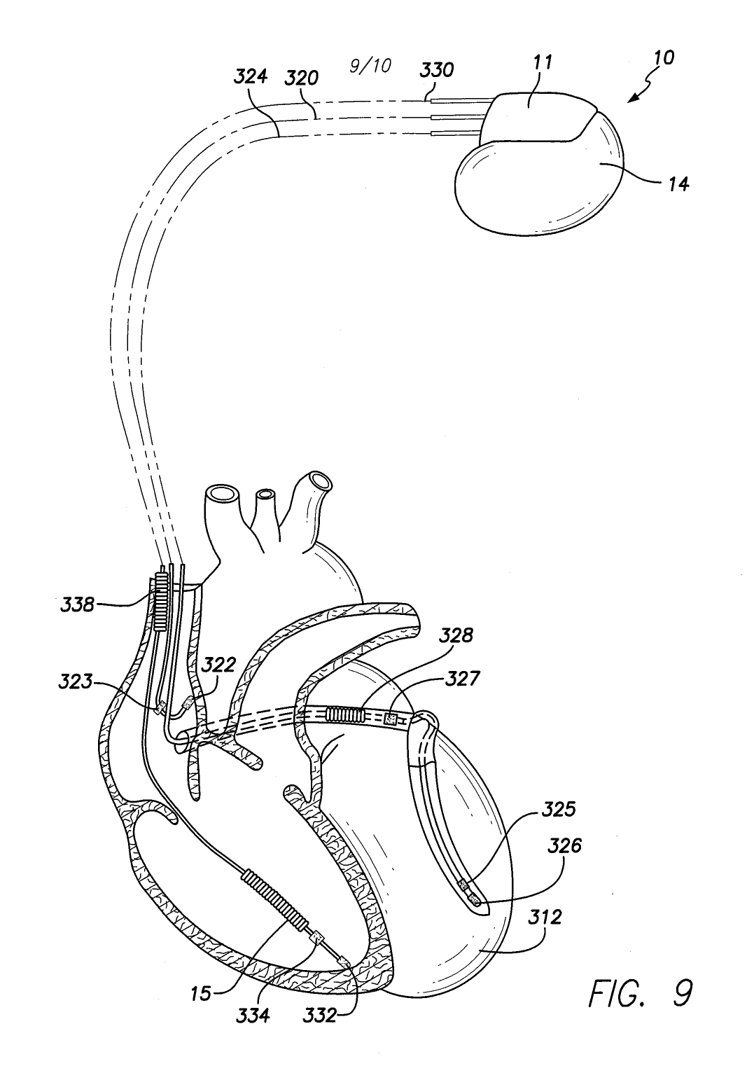

FIG. 1 illustrates an implantable medical system 8 having an ICD 10 equipped with internal MICS/MedRadio components and an internal conductive EMI noise shield (not shown in FIG. 1) and further equipped with one or more cardiac sensing/pacing/shocking leads 12 implanted within the heart of the patient. (The ICD may be equipped to perform both pacing and shocking functions and may also be referred to as a hybrid pacemaker/ICD or just a “hybrid.”) In FIG. 1, two exemplary leads are shown: a bipolar RV lead and a bipolar left ventricular (LV) lead implanted via the coronary sinus (CS). An RA lead may also provided that includes a bipolar RA tip/ring pair. Other suitable leads may instead be employed, including leads with more or fewer electrodes, such as quadripolar leads. Also, as shown, the exemplary RV lead has an RV coil 15 implanted within the RV for delivery of defibrillation shocks (while using the case 14 or housing or “can” of the ICD as a return electrode.) Other lead electrodes of various sizes and shapes may be additionally or alternatively provided, such as an LV coil. A more complete set of leads is illustrated in FIG. 9.

The internal MICS/MedRadio components of ICD 8 use case 14 of the ICD as part of the antenna for communicating with an external system 16 via MICS/MedRadio-band signals. External system 16 may include, for example, an external programmer, bedside monitor, base station or hand-held personal advisory module (PAM). The MICS/MedRadio components may exploit InvisiLink™ Wireless Telemetry of St. Jude Medical. For example, periodic transfers for diagnostics data may be transmitted from the ICD to a bedside monitor located within about two meters of the patient. Data from the external system can then be forwarded to a centralized system such as the Merlin.Net system, the HouseCall™ remote monitoring system or the Merlin@home systems of St. Jude Medical so as to relay the information to a clinician.

Although identified in FIG. 1 as an ICD, it should be understood that device 10 can be any suitably-equipped implantable medical device, such as a standalone pacemaker or CRT device, including CRT-D and CRT-P devices or other cardiac rhythm management devices (CRMDs.) The aforementioned internal EMI noise shield is particularly useful within defibrillation devices for attenuating electrical interference during MICS/MedRadio communication but might be useful in other devices as well, or for other purposes. ICDs are generally discussed, for example, in U.S. Pat. No. 5,720,767 to Amely-Velez, entitled “Impedance Dependent Implantable Cardioverter-Defibrillator.”

It should be understood that the particular shape, size and locations of the implanted components shown in FIG. 1 are merely illustrative and may not necessarily correspond to actual implant locations. In particular, preferred implant locations for the leads are more precisely illustrated in FIG. 9.

### Shielding Components

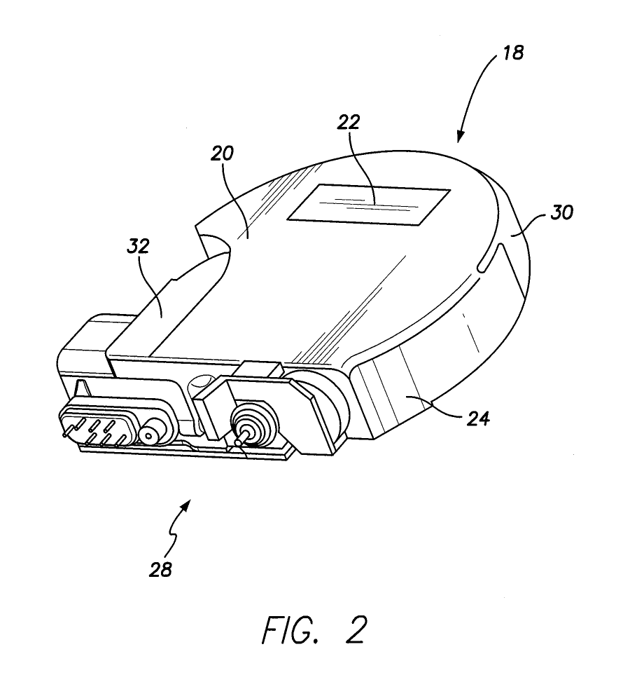

FIGS. 2-5 illustrate a conductive EMI noise shield that may be used within the ICD of FIG. 1 or other suitably equipped systems. Referring first to FIG. 2, a perspective view of a “top” of ICD 10 is shown without its case to illustrate noise shield 18. In this example, shield 18 has a first generally flat portion 20 for mounting along a first lateral (or top) side of the ICD. An adhesive 22 (which may be a piece of double stick foam tape) is used to adhere the top can of the ICD to the ICD assembly. There is an opening in the shield to allow adhesion of the tape to the battery and the top can. The tape is non-conductive. Shield portion 20 is located on top of the device's battery (not specifically shown in FIG. 2) and over the high voltage capacitors (also not specifically shown.) Shield 18 also includes a smaller side portion 24 for mounting along an outer edge or periphery of the ICD to provide continuity between the top and bottom portions of the shield.

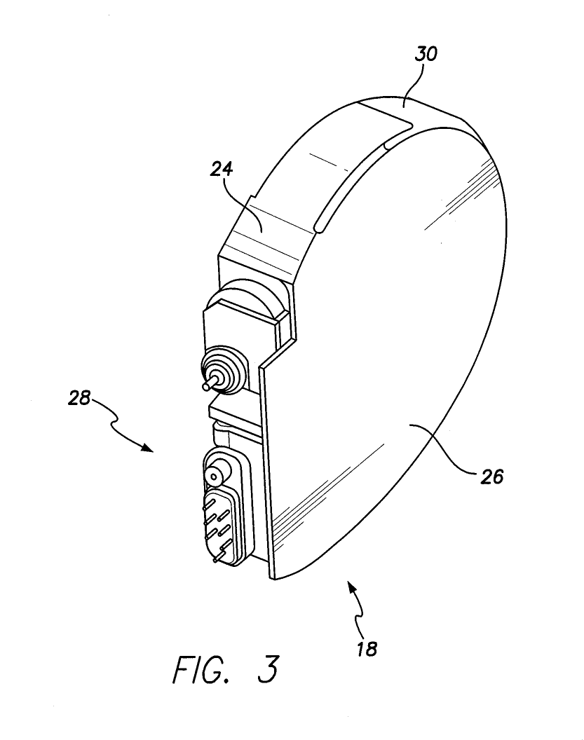

Referring to FIG. 3, shield 18 also includes a second generally flat portion 26 for mounting along a second lateral side (or “bottom”) of the ICD. (Within hybrid pacemaker/ICDs, this portion of the hybrid is connected to the lowest potential in the ICD. Also, it should be understood that the terms “top” and “bottom” and “first” and “second” are arbitrary designator terms herein and devices can be constructed wherein “top” and “bottom” or “first” and “second” are reversed or where other designator terms are used.) Hence, with the configuration of FIGS. 2 and 3, the shield wraps (or flexes) around the ICD to enclose or cover a large portion of the ICD and separate it from the housing. Note however that the flex shield does not enclose all of the ICD. In particular, a feedthrough end portion 28 is not covered to allow connection of leads to the ICD. Feedthroughs are discussed, for example, in U.S. Pat. No. 7,260,434 to Lim et al., entitled “Integrated 8-pole Filtered Feedthrough with Backfill Tube for Implantable Medical Devices.” Moreover, an opposing upper end 30 of the ICD is not covered. Additionally, a rectangular area 32 remains uncovered by the shield in the vicinity of the charger, a design choice which may provide some functional benefits.

Note that it would be generally preferable (in at least some devices) to provide a shield that encloses the entire inner surface of the housing of the ICD and therefore more fully isolates the antenna ground plane from the noise source. Practical considerations such as manufacturability and high volume production tend to lead to engineering choices that result in a shield with less coverage. However, extensive Bit Error Rate testing has shown that the shield of FIGS. 2 and 3 generally improves radio sensitivity and allows the system to more easily meet operational requirements. Although not shown in FIGS. 2 and 3, an insulation layer may be coated on shield 18 to help insulate the shield from internal electrical components of the ICD, particularly to prevent shorts with the high-voltage components during generation and delivery of defibrillation shocks.

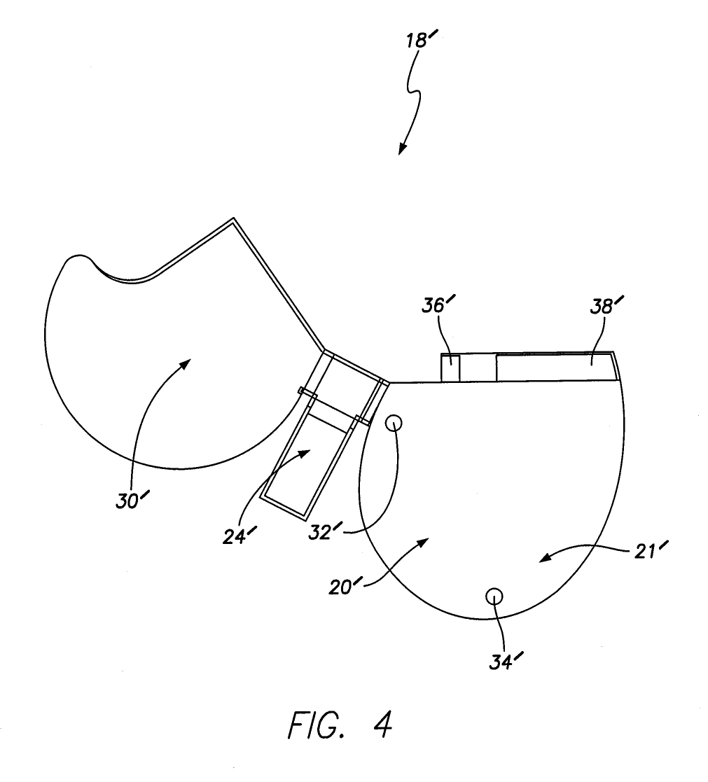

FIG. 4 illustrates a slightly different configuration of the shield, denoted by reference numeral 18′, which includes a pair of circular apertures 32′ and 34′ as well as flange portions 36′ and 38′. The circular apertures are location holes for a fixture used to install the shield. The flange portions provide shielding near the feedthrough where the leads are connected to the ICD. Note that, in this example, portions 24′ and 30′ are covered with a nonconductive adhesive, typically about 0.001 inches thick. Portion 20′ is covered by a conductive adhesive 21′, typically about 0.002 inches thick. The conducting adhesive may be a pressure sensitive adhesive formed with spherically-shaped beads (or other beads of suitable shape such as beads that might have a more cubic shape) provided to enhance electrical contact with the lowest potential node in the circuit (which is itself formed of metal or other conducting materials.) The spherically-shaped beads may be formed of copper with average sizes of about 1 or 2 mils. A suitable conducting adhesive may be obtained from 3M Inc.

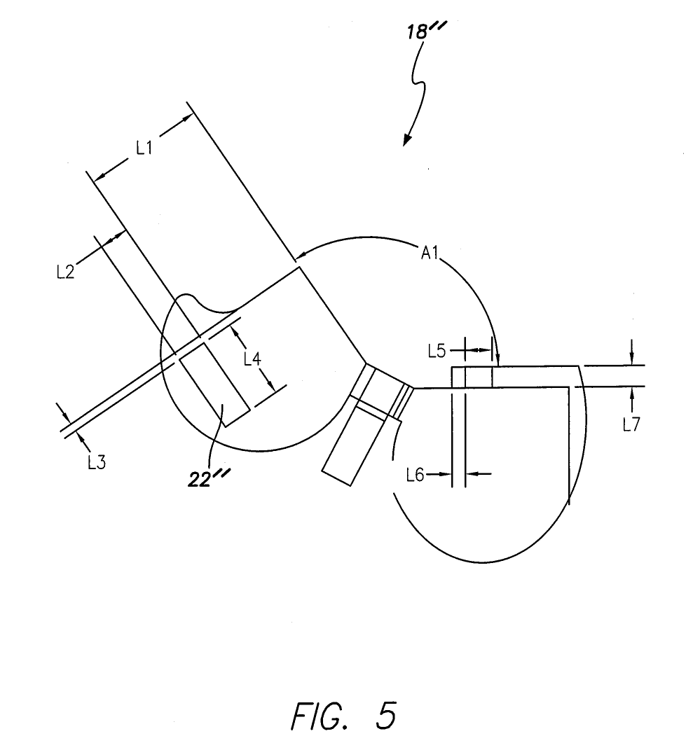

FIG. 5 illustrates yet another slightly different configuration of the shield, denoted by reference numeral 18″, which includes flange portions but no circular apertures. FIG. 5 also illustrates exemplary dimensional variables or values. In one example, the following length values are used (with all measurements in inches): L1 is 1.111; L2 is 0.275; L3 is 0.79; L4 is 0.75; L5 is 0.250; L6 is 0.125; and L7 is 0.175. Angle A1 is 124.2 degrees. These dimensions would differ for an ICD of a different size or shape. The copper portion of the shield is about 1-2 mils thick.

### Functional Schematic

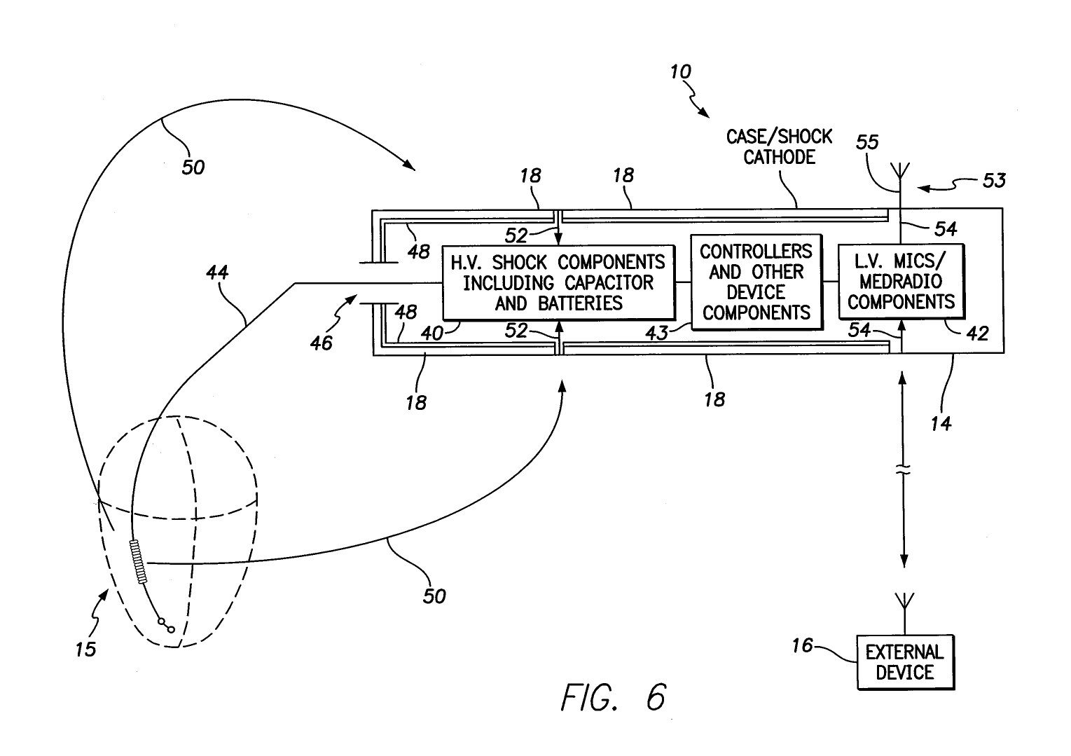

FIG. 6 is a functional schematic illustration of pertinent components of the ICD along with a stylized illustration of the heart. Within case or housing 14, high voltage shock components 40 and low voltage MICS/MedRadio components 42 are illustrated in block diagram form, along with other device components 43 (such as microcontrollers, etc.) The high voltage shock components (including the shock capacitor and various components to control its charge and discharge) are switchably connected to RV lead 44 via a feedthrough 46 for delivering a defibrillation shock using RV coil 15. The low voltage MICS/MedRadio components are connected directly to case 14 via capacitors (not shown in FIG. 6) for use as part of an antenna to communicate with external device 16. In one example, two capacitors are used. In the figure, a MICS/MedRadio antenna 53 is shown schematically. It should be understood that the antenna actually comprises the case and a radiative wire that is contained inside the header of the ICD. To show the radiative wire, a portion 55 of the antenna is identified in the figure. In a practical implementation, this portion is mounted within the header (which is the portion of device 10 of FIG. 9 to which the leads are attached.) To deliver a defibrillation shock, a high voltage circuit is connected to allow current to pass from the shock capacitor through the RV coil and then back to case 14 of the ICD via tissue propagation pathways 50. The return current then passes from the can to the high voltage capacitor via internal connection lines 52, while bypassing conductive shield 18 and insulation layers 48. That is, the shock current does not flow through the shield. (Note that if the polarity were reversed, the current would flow in the opposite direction.) Note that the MICS/MedRadio components are always connected to case 14 via interconnection lines 54 (which are connected to appropriate electrical terminals of the internal MICS/MedRadio components via one or more capacitors) and so radio communication need not be suspended during delivery of a shock. While the MICS/MedRadio components are using the case as part of the antenna, EMI noise shield 18 attenuates electrical noise emanating from the high voltage components to reduce or minimize interference with the antenna of which the case is a part. As already explained, the shield does not necessarily encompass the entire inner surface of the case to allow access to the case by the MICS/MedRadio components or other components. In any case, the shield is located in a manner that prevents noise from certain components to reach one of the elements of the antenna, which is the case. For the sake of completeness, additional information regarding the shield and its function will now be provided by way of figures generally directed to assembly and usage techniques.

### EMI Noise Shield Assembly and Usage Techniques

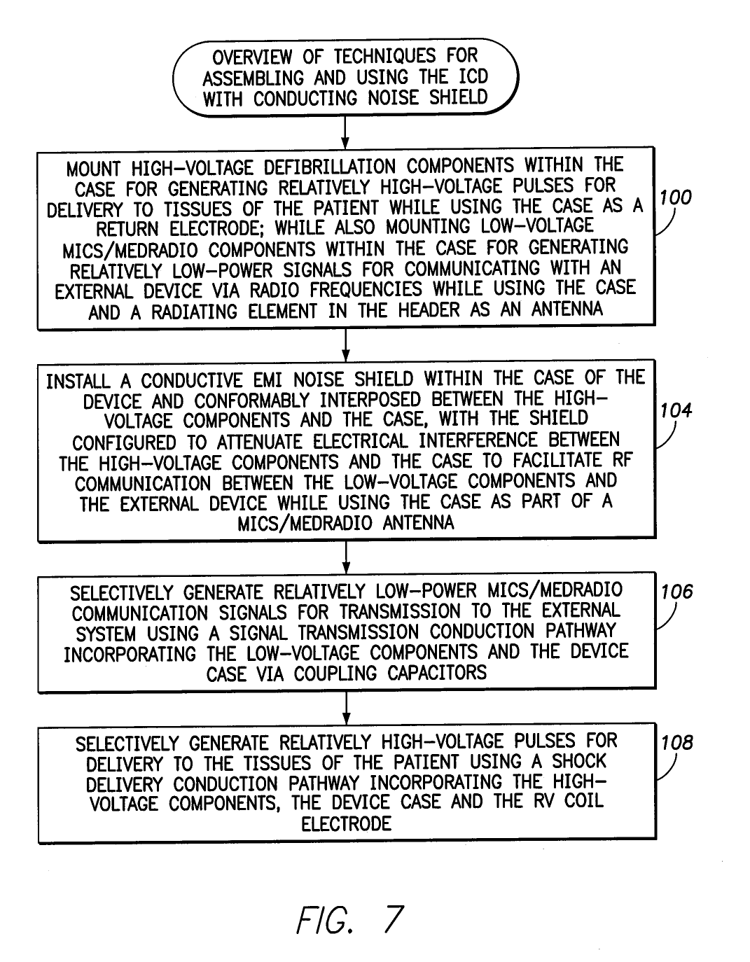

FIG. 7 provides an overview of techniques appropriate for use with the conductive noise shield. At step 100, high-voltage defibrillation components are mounted within the case of the ICD for generating relatively high-voltage pulses for delivery to tissues of the patient while using the case as a stimulation electrode. Also, during step 100, low-voltage MICS/MedRadio components are mounted within the case for generating relatively low-power signals for communicating with the external device via RF while using the case and a radiating element within the header as an antenna. At step 104, the conductive EMI noise shield is installed within the case of the device and conformably interposed between the high-voltage components and the case, with the shield configured to attenuate electrical interference between the high-voltage components and the case to facilitate radio-frequency communication between the low-voltage components and the external device while the low-voltage components use the case as part of the antenna (along with a radiating element in the header.) Note that the entire device is built at once and then the shield is installed. Once the shield is installed, the device in placed inside the can. Note also that methods of manufacturing implantable medical devices are discussed in U.S. Pat. No. 7,729,769 to Xie et al., entitled “Implantable Medical Device with Improved Back-Fill Member and Methods of Manufacture Thereof.”

At step 106, the ICD (following implant into a patient) selectively generates relatively low-power communication signals (such as MICS/MedRadio-based signals) for transmission to the external system using a signal transmission conduction pathway incorporating the low-voltage components and the device case as part of the antenna (via various coupling capacitors.) The noise shield is not part of the signal transmission conduction pathway. The MICS/MedRadio-based signals may be transmitted using RF frequencies around 400 MHz. At step 108, the device selectively generates relatively high-voltage pulses for delivery to the tissues of the patient using a shock delivery conduction pathway incorporating the high-voltage components, the device case and an electrode within an implantable stimulation lead. The EMI noise shield is not part of the shock delivery conduction pathway either. Rather, the EMI shield is positioned and configured to attenuate high voltage noise (particularly electrical components thereof) to prevent that noise from significantly interfering with MICS/MedRadio communications.

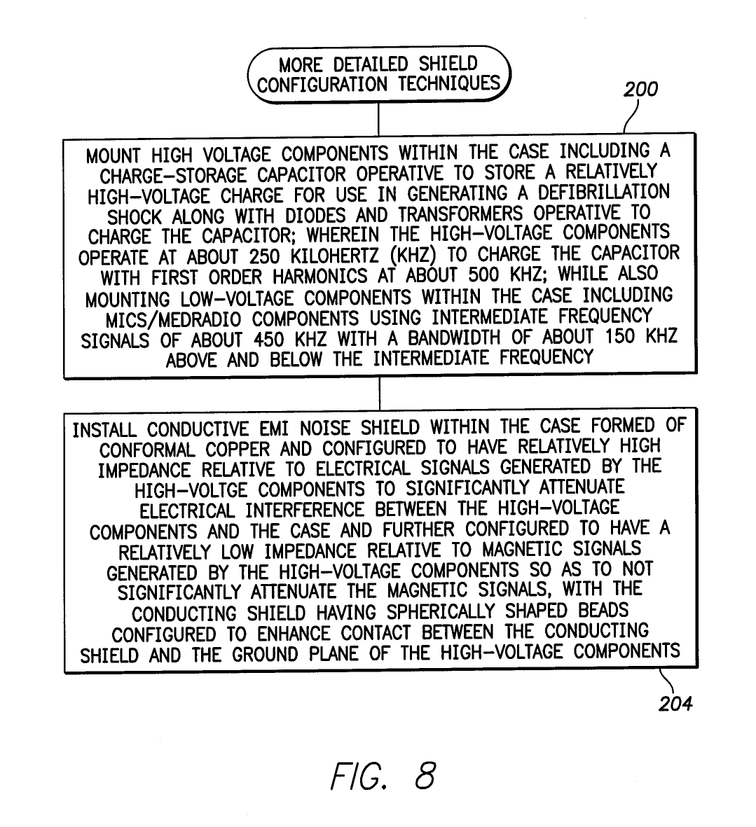

FIG. 8 provides further details pertaining to some of the aspects of FIG. 7. Insofar as the high voltage components are concerned, at step 200, high-voltage components are mounted within the case, including a charge-storage capacitor operative to store a relatively high-voltage charge for use in generating a defibrillation shock along with diodes and transformers operative to charge the capacitor, wherein the high-voltage components operate at about 250 kHz to charge the capacitor with first order harmonics at about 500 kHz. Also, at step 200, the low-voltage components are mounted within the case, including MICS/MedRadio components using intermediate frequency signals of about 450 kHz with a bandwidth of about 150 kHz above and below the intermediate frequency. At step 204, the conductive EMI noise shield is installed within the case wherein the shield is formed of conformal copper and configured to have relatively high impedance relative to electrical signals generated by the high-voltage components to significantly attenuate electrical interference between the high-voltage components and the case. The shield is further configured to have a relatively low impedance relative to magnetic signals generated by the high-voltage components and does not significantly attenuate the magnetic signals. As already explained, the conductive shield may be provided with spherically-shaped beads configured to enhance contact between a portion of the conductive shield and the ground plane of the high-voltage components (which may be the bare metal “back” of the hybrid ICD.)

Hence, insofar as the frequencies are concerned, the MICS/MedRadio components in this example operate using IF signals that correspond to about twice the fundamental charger frequency (and hence might detect unwanted harmonics produced by the high voltage components of the charger if no shield were provided.) It is noted that this need not be the case in all devices. In at least some examples, though, for design legacy reasons or for other reasons, the frequencies line up resulting in possible noise problems, which the shield described herein addresses. Also, note that the exemplary frequency numbers, physical dimensions, and other values presented herein may be typical but are not fully representative of the broad range of values that an engineer would experience or use in implementing the invention.

Insofar as impedance is concerned, the conformal shield is configured to attenuate noise from the high-voltage components by exploiting impedance discontinuities between the emissions of the noisy high-voltage components and the shield. As already noted, within an ICD, the source of noise is mostly electrical in nature. Due to the thinness and permeability of the copper metal employed in the conformal shield, the shield provides very little attenuation to magnetic noise sources because the low impedance of the magnetic waveform closely matches the low impedance of the copper material. Hence, closely-matched impedances allow the magnetic waves to pass through the copper metal with very little attenuation, which may be of benefit in avoiding inadvertent shielding of any magnetic sensors mounted within the device. However, the electric field wave has very high impedance compared to the shield metal and so much of the incident waveform is reflected and prevented from reaching the antenna ground plane. That is, for noise signals that are of high impedance nature (high voltage, low current sourced), the shield represents a low impedance and due to this mismatch in impedances, there is a relatively large reflection that results in a shielding effect. In the case of low impedance signals (low voltage, high current sourced), the impedance of the signal and the impedance of the shield are both low and therefore there is very little reflection. In this regard, copper is not a very good magnetic shield material (but iron is a good magnetic shield material.)

Although primarily described with respect to examples wherein the implanted device is an ICD, other implantable medical devices may be equipped to exploit the techniques described herein. For the sake of completeness, an exemplary ICD will now be described, which includes components for performing controlling pacing and shocking.

### Exemplary ICD

FIG. 9 provides a simplified block diagram of the ICD, which in this example is a dual-chamber hybrid device capable of treating both fast and slow arrhythmias with stimulation therapy, including cardioversion, defibrillation, and pacing stimulation. (A single chamber ICD could instead be used.) To provide atrial chamber pacing stimulation and sensing, ICD 10 is shown in electrical communication with a heart 312 by way of a right atrial lead 320 having an atrial tip electrode 322 and an atrial ring electrode 323 implanted in the atrial appendage. ICD 10 is also in electrical communication with the heart by way of a right ventricular lead 330 having, in this embodiment, a ventricular tip electrode 332, a right ventricular ring electrode 334, a right ventricular (RV) coil electrode 15, and a superior vena cava (SVC) coil electrode 338. Typically, the right ventricular lead 330 is transvenously inserted into the heart so as to place the RV coil electrode 15 in the right ventricular apex, and the SVC coil electrode 338 in the superior vena cava. Accordingly, the right ventricular lead is capable of receiving cardiac signals, and delivering stimulation in the form of pacing and shock therapy to the right ventricle.

To sense left atrial and ventricular cardiac signals and to provide left chamber pacing therapy, ICD 10 is coupled to a “coronary sinus” lead 324 designed for placement in the “coronary sinus region” via the coronary sinus os for positioning a distal electrode adjacent to the left ventricle and/or additional electrode(s) adjacent to the left atrium. As used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus. Accordingly, an exemplary coronary sinus lead 324 is designed to receive atrial and ventricular cardiac signals and to deliver left ventricular pacing therapy using at least a left ventricular tip electrode 326, left atrial pacing therapy using at least a left atrial ring electrode 327, and shocking therapy using at least a left atrial coil electrode 328. With this configuration, biventricular pacing can be performed. Although only three leads are shown in FIG. 9, it should also be understood that additional stimulation leads (with one or more pacing, sensing and/or shocking electrodes) might be used in order to efficiently and effectively provide pacing stimulation to the left side of the heart or atrial cardioversion and/or defibrillation. A portion 11 of ICD 10 represents the header of the device (to which the leads are connected.) Within the header, the aforementioned radiating wire is mounted, which forms a part of the radio antenna.

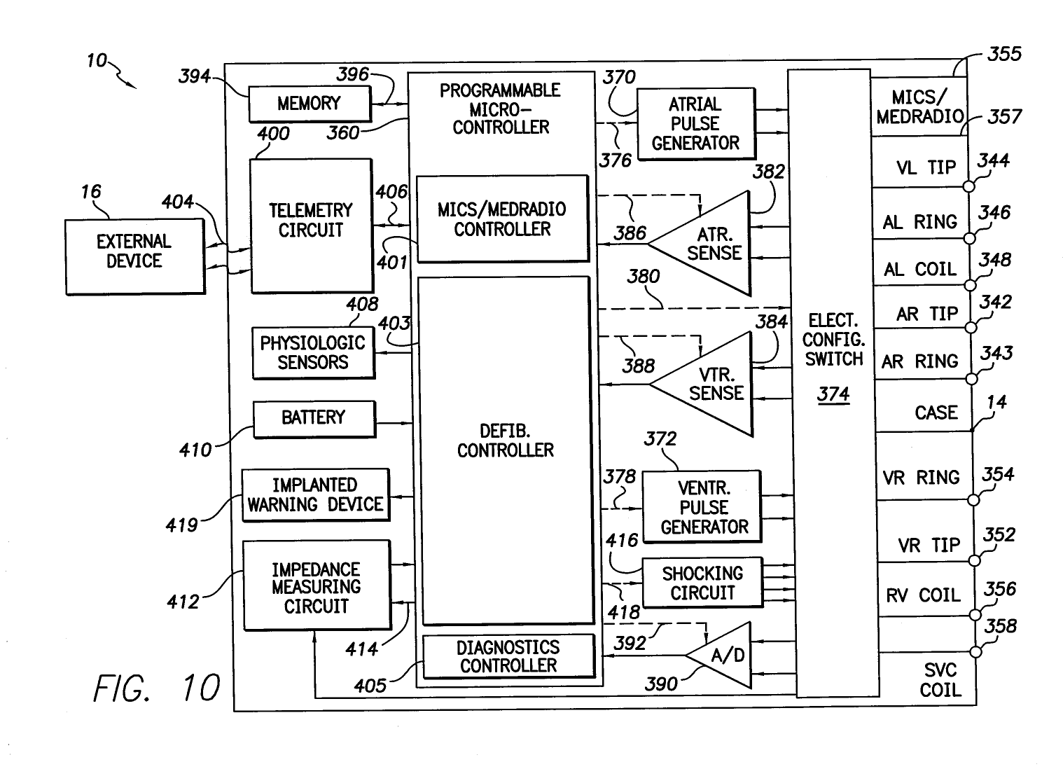

A simplified block diagram of internal components of ICD 10 is shown in FIG. 10. While a particular ICD is shown, this is for illustration purposes only, and one of skill in the art could readily duplicate, eliminate or disable the appropriate circuitry in any desired combination to provide a device capable of treating the appropriate chamber(s) with cardioversion, defibrillation and pacing stimulation.

The housing 14 for ICD 10, wherein the housing is shown schematically in FIG. 10, is often referred to as the “can”, “case” or “case electrode” and may be programmably selected to act as the return electrode for all “unipolar” modes. The housing 14 may further be used as a return electrode alone or in combination with one or more of the coil electrodes, 328, 15 and 338, for shocking purposes. Note that the diagram of FIG. 10 does not illustrate the aforementioned noise shield, which is illustrated within figures already described. The housing 14 includes a connector (not shown) having a plurality of terminals, 342, 343, 344, 346, 348, 352, 354, 356 and 358 (shown schematically and, for convenience, the names of the electrodes to which they are connected are shown next to the terminals). As such, to achieve right atrial sensing and pacing, the connector includes at least a right atrial tip terminal (A_{R} TIP) 342 adapted for connection to the atrial tip electrode 322 and a right atrial ring (A_{R} RING) electrode 343 adapted for connection to right atrial ring electrode 323. To achieve left chamber sensing, pacing and shocking, the connector includes at least a left ventricular tip terminal (V_{L} TIP) 344, a left atrial ring terminal (A_{L} RING) 346, and a left atrial shocking terminal (A_{L} COIL) 348, which are adapted for connection to the left ventricular ring electrode 326, the left atrial tip electrode 327, and the left atrial coil electrode 328, respectively. To support right chamber sensing, pacing and shocking, the connector further includes a right ventricular tip terminal (V_{R} TIP) 352, a right ventricular ring terminal (V_{R} RING) 354, a right ventricular shocking terminal (Rv COIL) 356, and an SVC shocking terminal (SVC COIL) 358, which are adapted for connection to the right ventricular tip electrode 332, right ventricular ring electrode 334, the RV coil electrode 15, and the SVC coil electrode 338, respectively. Still further, MICS/MedRadio interconnection lines 355 and 357 are provided for connection to the housing to allow MICS/MedRadio components to use the housing (or case) as part of the antenna, as already explained.

At the core of ICD 10 is a programmable microcontroller 360, which controls the various modes of stimulation therapy. As is well known in the art, the microcontroller 360 (also referred to herein as a control unit) typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 360 includes the ability to process or monitor input signals (data) as controlled by a program code stored in a designated block of memory. The details of the design and operation of the microcontroller 360 are not critical to the invention. Rather, any suitable microcontroller 360 may be used that carries out the functions described herein. The use of microprocessor-based control circuits for performing timing and data analysis functions are well known in the art.

As shown in FIG. 10, an atrial pulse generator 370 and a ventricular/impedance pulse generator 372 generate pacing stimulation pulses for delivery by the right atrial lead 320, the right ventricular lead 330, and/or the coronary sinus lead 324 via an electrode configuration switch 374. It is understood that in order to provide stimulation therapy in each of the four chambers of the heart, the atrial and ventricular pulse generators, 370 and 372, may include dedicated, independent pulse generators, multiplexed pulse generators or shared pulse generators. The pulse generators, 370 and 372, are controlled by the microcontroller 360 via appropriate control signals, 376 and 378, respectively, to trigger or inhibit the stimulation pulses.

The microcontroller 360 further includes timing control circuitry (not separately shown) used to control the timing of such stimulation pulses (e.g., pacing rate, atrio-ventricular (AV) delay, atrial interconduction (A-A) delay, or ventricular interconduction (V-V) delay, etc.) as well as to keep track of the timing of refractory periods, blanking intervals, noise detection windows, evoked response windows, alert intervals, marker channel timing, etc., which is well known in the art. Switch 374 includes a plurality of switches for connecting the desired electrodes to the appropriate I/O circuits, thereby providing complete electrode programmability. Accordingly, the switch 374, in response to a control signal 380 from the microcontroller 360, determines the polarity of the stimulation pulses (e.g., unipolar, bipolar, combipolar, etc.) by selectively closing the appropriate combination of switches (not shown) as is known in the art.

Atrial sensing circuits 382 and ventricular sensing circuits 384 may also be selectively coupled to the right atrial lead 320, coronary sinus lead 324, and the right ventricular lead 330, through the switch 374 for detecting the presence of cardiac activity in each of the four chambers of the heart. Accordingly, the atrial (ATR. SENSE) and ventricular (VTR. SENSE) sensing circuits, 382 and 384, may include dedicated sense amplifiers, multiplexed amplifiers or shared amplifiers. The switch 374 determines the “sensing polarity” of the cardiac signal by selectively closing the appropriate switches, as is also known in the art. In this way, the clinician may program the sensing polarity independent of the stimulation polarity. Each sensing circuit, 382 and 384, preferably employs one or more low power, precision amplifiers with programmable gain and/or automatic gain control, bandpass filtering, and a threshold detection circuit, as known in the art, to selectively sense the cardiac signal of interest. The automatic gain control enables ICD 10 to deal effectively with the difficult problem of sensing the low amplitude signal characteristics of atrial or ventricular fibrillation. The outputs of the atrial and ventricular sensing circuits, 382 and 384, are connected to the microcontroller 360 which, in turn, are able to trigger or inhibit the atrial and ventricular pulse generators, 370 and 372, respectively, in a demand fashion in response to the absence or presence of cardiac activity in the appropriate chambers of the heart.

For arrhythmia detection, ICD 10 utilizes the atrial and ventricular sensing circuits, 382 and 384, to sense cardiac signals to determine whether a rhythm is physiologic or pathologic. As used in this section, “sensing” is reserved for the noting of an electrical signal, and “detection” is the processing of these sensed signals and noting the presence of an arrhythmia. The timing intervals between sensed events (e.g., P-waves, R-waves, and depolarization signals associated with fibrillation which are sometimes referred to as “F-waves” or “Fib-waves”) are then classified by the microcontroller 360 by comparing them to a predefined rate zone limit (i.e., bradycardia, normal, atrial tachycardia, atrial fibrillation, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, antitachycardia pacing, cardioversion shocks or defibrillation shocks).

Cardiac signals are also applied to the inputs of an analog-to-digital (A/D) data acquisition system 390. The data acquisition system 390 is configured to acquire intracardiac electrogram signals, convert the raw analog data into a digital signal, and store the digital signals for later processing and/or telemetric transmission to an external device 402. The data acquisition system 390 is coupled to the right atrial lead 320, the coronary sinus lead 324, and the right ventricular lead 330 through the switch 374 to sample cardiac signals across any pair of desired electrodes. The microcontroller 360 is further coupled to a memory 394 by a suitable data/address bus 396, wherein the programmable operating parameters used by the microcontroller 360 are stored and modified, as required, in order to customize the operation of ICD 10 to suit the needs of a particular patient. Such operating parameters define, for example, pacing pulse amplitude or magnitude, pulse duration, electrode polarity, rate, sensitivity, automatic features, arrhythmia detection criteria, and the amplitude, waveshape and vector of each shocking pulse to be delivered to the patient's heart within each respective tier of therapy. Other pacing parameters include base rate, rest rate and circadian base rate.

Advantageously, the operating parameters of the implantable ICD 10 may be non-invasively programmed into the memory 394 through a telemetry circuit 400 in telemetric communication with the external device 402, such as a programmer, transtelephonic transceiver or a diagnostic system analyzer. The telemetry circuit 400 is activated by the microcontroller by a control signal 406. The telemetry circuit 400 advantageously allows intracardiac electrograms and status information relating to the operation of ICD 10 (as contained in the microcontroller 360 or memory 394) to be sent to the external device 402 through an established communication link 404. Depending upon the implementation, the telemetry circuit may exploit MICS/MedRadio components to facilitate telemetry. ICD 10 further includes an accelerometer or other physiologic sensor 408, commonly referred to as a “rate-responsive” sensor because it is typically used to adjust pacing stimulation rate according to the exercise state of the patient. However, physiological sensor 408 may further be used to detect changes in cardiac output, changes in the physiological condition of the heart, or diurnal changes in activity (e.g., detecting sleep and wake states) and to detect arousal from sleep. Accordingly, microcontroller 360 responds by adjusting the various pacing parameters (such as rate, AV Delay, V-V Delay, etc.) at which the atrial and ventricular pulse generators, 370 and 372, generate stimulation pulses. While shown as being included within ICD 10, it is to be understood that physiologic sensor 408 may also be external to ICD 10 yet still be implanted within or carried by the patient. A common type of rate responsive sensor is an activity sensor incorporating an accelerometer or a piezoelectric crystal, mounted within the housing of the ICD. Other types of physiologic sensors are known, for example, sensors that sense the oxygen content of blood, respiration rate and/or minute ventilation, pH of blood, ventricular gradient, pulmonary artery pressure, etc.

The ICD additionally includes a battery 410, which provides operating power to all of the circuits shown in FIG. 10. The battery 410 may vary depending on the capabilities of ICD 10. If the system only provides low voltage therapy, a lithium iodine or lithium copper fluoride cell may be utilized. For ICD 10, which employs shocking therapy, the battery 410 must be capable of operating at low current drains for long periods, and then be capable of providing high-current pulses (for capacitor charging) when the patient requires a shock pulse. The battery 410 must also have a predictable discharge characteristic so that elective replacement time can be detected. Accordingly, ICD 10 is preferably capable of high voltage therapy and appropriate batteries.

As further shown in FIG. 10, ICD 10 is shown as having an impedance measuring circuit 412, which is enabled by the microcontroller 360 via a control signal 414. The impedance circuit may be used for detecting thoracic and/or cardiogenic impedance. Other uses for an impedance measuring circuit include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring stroke volume; detecting the opening of heart valves. The impedance measuring circuit 412 is advantageously coupled to the switch 374 so that any desired electrode may be used.

In the case where ICD 10 is intended to operate as an implantable cardioverter/defibrillator (ICD) device, it detects the occurrence of an arrhythmia, and automatically applies an appropriate electrical shock therapy to the heart aimed at terminating the detected arrhythmia. To this end, the microcontroller 360 further controls a high voltage shocking circuit 416 by way of a control signal 418. The shocking circuit 416 generates shocking pulses of low (up to 0.5 joules), moderate (0.5-10 joules) or high energy (11 to 40 joules or more), as controlled by the microcontroller 360. Such shocking pulses are applied to the heart of the patient through at least two shocking electrodes, and as shown in this embodiment, selected from the left atrial coil electrode 328, the RV coil electrode 15, and/or the SVC coil electrode 338. The housing 14 may act as an active electrode in combination with the RV electrode 15, or as part of a split electrical vector using the SVC coil electrode 338 or the left atrial coil electrode 328 (i.e., using the RV electrode as a common electrode). Cardioversion shocks are generally considered to be of low to moderate energy level (so as to minimize pain felt by the patient), and/or synchronized with an R-wave and/or pertaining to the treatment of tachycardia. Defibrillation shocks are generally of moderate to high energy level (i.e., corresponding to thresholds in the range of 3-40 joules or more), delivered asynchronously (since R-waves may be too disorganized), and pertaining exclusively to the treatment of fibrillation. Accordingly, the microcontroller 360 is capable of controlling the synchronous or asynchronous delivery of the shocking pulses.

Microcontroller 360 also includes various components directed to controlling MICS/MedRadio communication, defibrillation and diagnostics. Briefly, a MICS/MedRadio controller 401 controls MICS/MedRadio communications, as described above. (Additional MICS/MedRadio components may also be provided within the device such as the radiative wire mounted within the header of the device.) A defibrillation controller 403 controls delivery of a defibrillation shock. Diagnostics pertinent to MICS/MedRadio communications, defibrillation or any other functions of the device may be generated under the control of diagnostics controller 405 for storage within memory 394 for transfer to an external device.

Depending upon the implementation, the various components of the microcontroller of the implanted device may be implemented as separate software modules or the modules may be combined to permit a single module to perform multiple functions. In addition, although shown as being components of the microcontroller, some or all of these components may be implemented separately from the microcontroller, using application specific integrated circuits (ASICs) or the like.

In general, while the invention has been described with reference to particular embodiments, modifications can be made thereto without departing from the scope of the invention. Note also that the term “including” as used herein is intended to be inclusive, i.e. “including but not limited to.”

What is claimed is:

1. An implantable medical device for implant within a patient, the device comprising:
high-voltage components operative to generate relatively high-voltage pulses for delivery to tissues of the patient while using a case of the device as a stimulation electrode;
low-voltage components operative to generate relatively low-power signals for communicating with an external device while using the case as part of an antenna; and
a conductive shield interposed between the high-voltage components and the case and configured to attenuate electrical interference between the high-voltage components and the case to facilitate communication between the low-power components and the external device while using the case as part of the antenna.

2. The device of claim 1 wherein the implantable medical device is an implantable cardioverter-defibrillator (ICD) and wherein the high-voltage components include a charge-storage capacitor operative to store a relatively high-voltage charge for use in generating a defibrillation shock.

3. The device of claim 2 wherein the high-voltage components are configured to operate at about 250 kilohertz (KHz) to charge the capacitor with first order harmonics at about 500 KHz.

4. The device of claim 1 wherein the low-power components operate using intermediate frequency signals of about 450 KHz with a bandwidth of about 150 KHz above and below the intermediate frequency.

5. The device of claim 4 wherein the low-power components include one or more of Medical Implant Communication Service (MICS) components and Medical Device Radiocommunications Service (MedRadio) components.

6. The device of claim 1 wherein the shield has relatively high impedance relative to electrical signals generated by the high-voltage components so as to significantly attenuate electrical interference between the high-voltage components and the case.

7. The device of claim 6 wherein the conductive shield has relatively low impedance relative to magnetic signals generated by the high-voltage components so as to not significantly attenuate the magnetic signals.

8. The device of claim 1 wherein the conductive shield is conformably interposed between the high-voltage components and adjacent interior surfaces of the case to attenuate electrical interference between the high-voltage components and the case.

9. The device of claim 1 wherein the conductive shield includes a first shielding portion near a first interior portion of the case via a non-conducting adhesive.

10. The device of claim 1 wherein the conductive shield includes a second shielding portion near a second interior portion of the case and connected to a lowest potential ground plane of the high-voltage components of the device via an electrical contact.

11. The device of claim 10 wherein the electrical contact is a conductive adhesive.

12. The device of claim 10 wherein the conducting adhesive includes beads configured to enhance contact between the first portion of the conductive shield and the ground plane of the high-voltage components.

13. The device of claim 1 wherein the conductive shield has an insulating layer interposed between an inner surface of the conductive shield and internal electrical components of the device.

14. The device of claim 1 wherein the conductive shield is formed of conformal copper.

15. A method for use with an implantable medical device for implant within a patient, the device having high-voltage components for generating relatively high-voltage pulses for delivery to tissues of the patient while using a case of the device as a stimulation electrode and having low-voltage components for generating relatively low-power signals for communicating with an external device while using the case as part of an antenna, the method comprising:
mounting a conductive shield within the case of the device and interposed between the high-voltage components and the case, with the shield configured to attenuate electrical interference between the high-voltage components and the case to facilitate communication between the low-voltage components and the external device while using the case as part of an antenna;
selectively generating relatively high-voltage pulses for delivery to the tissues of the patient using a shock delivery conduction pathway incorporating the high-voltage components, the device case and an electrode of an implantable stimulation lead; and
selectively generating relatively low-power communication signals for transmission to the external system using a signal transmission conduction pathway incorporating the low-voltage components and the device case as part of the antenna.
